(19) United States
(12) Patent Application Publication
(10) Pub. No.: US 2025/0275676 A1
(43) Pub. Date: Sep. 4, 2025

(54) Visual Field Sensitivity Testing

(71) Applicants: University College Cardiff Consultants Limited, Cardiff (GB); University of Ulster, Belfast (GB); UCL Business Limited, London (GB)

(72) Inventors: Anthony William Redmond, Cardiff (GB); Pádraig joseph Mulholland, Belfast (GB); Roger Sproule Anderson, Belfast (GB); David Fitzgerald Garway-Health, London (GB)

(21) Appl. No.: 18/850,422

(22) PCT Filed: Mar. 31, 2023
(86) PCT No.: PCT/GB2023/050860

§ 371 Date: Sep. 24, 2024

(30) Foreign Application Priority Data
Jan. 4, 2022 (GB) 2204785.6

(51) Int. Cl. A61B 3/024

(52) U.S. Cl. CPC A61B 3/024

(57) ABSTRACT

The present invention is concerned with a method and an instrument for measuring visual field sensitivity wherein the method relies on measuring, across a subject's visual field, the subject's response to a fixed-luminance stimulus, the area of which is modulated on subsequent stimulation; and said instrument is adapted to emit, at one or more locations across a subject's visual field, a fixed-luminance stimulus whose area is modulated on subsequent stimulation and wherein the instrument is also adapted to record the subject's response to said fixed-luminance stimulus by comparing the subject's response with the response in an age-matched normal group or by comparing the response to a prior determined baseline in order to identify any change in visual field sensitivity.

### RELATED APPLICATIONS

This application is the national stage of international patent application no. PCT/GB2023/050860 filed on Mar. 31, 2023, which in turn claims priority from Great Britain Patent Application No. 2204785.6 filed on Apr. 1, 2022, the disclosures of which are incorporated herein by reference in their entirety.

### TECHNICAL FIELD

The present invention is concerned with a method and an instrument for measuring visual field sensitivity wherein the method relies on measuring, across a subject's visual field, the subject's response to a fixed-luminance stimulus, the area of which is modulated on subsequent stimulation; and said instrument is adapted to emit, at one or more locations across a subject's visual field, a fixed-luminance stimulus whose area is modulated on subsequent stimulation and wherein the instrument is also adapted to record the subject's response to said fixed-luminance stimulus by comparing the subject's response with the response in an age-matched normal group or by comparing the response to a prior determined baseline in order to identify any change in visual field sensitivity.

### BACKGROUND

Visual pathway disorder is characterised by one or more anomalies of the form and/or function of the structures of the visual pathway; a network of neurons that propagates visual signals within the eye, from the eye to the brain, and within the brain, as well as the non-neural structures that support neural structure and function. Visual pathway disorder can manifest as a disorder of visual perception or processing of visual stimuli, with form and function determined by the location and severity of the anomaly or anomalies along the visual pathway. Vision disorder is characterised by one or more anomalies of the form and/or function of the structures that make up the visual pathway.

Glaucoma, for example, is characterized by one or more of the following three features: 1) damage to retinal ganglion cells, 2) high intraocular pressure (IOP), and 3) progressive, often irreversible, loss of visual field sensitivity. The treatment for glaucoma is aimed at reducing intraocular pressure by therapeutic (eyedrops) or surgical means. Glaucoma is diagnosed from findings of three broad investigations: 1) imaging of the retina (optic nerve photographs and/or retinal layer scans), 2) measurement of IOP, and 3) measurement of visual field sensitivity.

Given that the ultimate goal of diagnosis and timely treatment is to halt and/or prevent vision loss, and that the prognosis for vision is best the earlier that changes are detected, it is important to obtain an accurate and precise measure of visual field sensitivity every time a subject is tested, and to be able to identify the subtlest signs of loss and deterioration of visual field sensitivity at the earliest possible opportunity.

Age-related macular degeneration (AMD) is an eye-disease that reduces the number and/or function of cells essential for vision in the central retina. It is the primary cause of visual impairment in the UK and can lead to marked reductions in quality-of-life in those with the condition. The measurement of visual function is fundamentally important to the detection and management of AMD.

One technique that is commonly used to measure visual function in glaucoma and AMD is perimetry.

Perimetry is the clinical method for measuring visual field sensitivity. It is commonly carried out on patients who are at risk of having eye disease, such as glaucoma. In those at risk and those suspected of having glaucoma, the primary aim is to identify differences in the visual field from that of an age-matched healthy cohort and/or from a baseline measure; visual field sensitivity that is below the range of normal values from healthy individuals (or that falls outside of baseline test-retest limits) increases the likelihood that that patient has glaucoma. Perimetry results are not considered in isolation, however, and are usually considered together with findings from retinal imaging and the IOP measurement.

Early perimeters involved moving small circular lights from outside the visual field into the central visual field and recording the point at which the patient could first see them. A series of isopters, representing isosensitivity contours, was plotted to denote the extent of the visual field. This is known as manual kinetic perimetry, and while it is still commonly used in the investigation of dense neurological defects such as stroke, it is now rarely used in the management of glaucoma. Instead, automated perimetry is used, where a 0.43 degrees diameter spot (fixed) varying in luminance has become the standard stimulus and is still used in the majority of visual field instruments today (e.g., the Humphrey Field Analyzer, Henson perimeters, Octopus perimeters).

Standard Automated Perimetry (SAP) measures visual field sensitivity by presenting fixed-area (Goldmann III) luminance-modulated stimuli to multiple locations in the visual field and recording the response of the patient; the patient presses a button if the stimulus is seen and does not press the button if the stimulus is not seen. Depending on the response of the patient for a given location, the luminance of the stimulus is adjusted until a determination can be made as to where the limit of visual detection lies. The limit of vision at each location is known as ‘threshold’. In clinical psychophysics literature, the term ‘sensitivity’ is commonly used, which is equivalent to 1/threshold. An example of the use of standard/conventional perimetry, where fixed-area (Goldmann III) luminance-modulated stimuli is presented to multiple locations in the visual field, is given in WO2014/094035.

A mathematical algorithm (thresholding algorithm) is used in the instrument to decide the next luminance value to present, based on responses of the patient to the previously presented stimuli. Typically, different instruments/tests use different thresholding algorithms. In the reference standard instrument (Humphrey Field Analyzer, Carl Zeiss Meditec, Dublin, CA, USA), the most common algorithm is known as the Swedish Interactive Thresholding Algorithm (SITA).

Once sensitivity is determined at all test locations, they are compared with normal ranges at the same locations from a normative database. Deviations from age-matched normal are calculated and statistical analysis is used to determine the probability that a total deviation at a given location is due to chance.

A further calculation is performed to account for diffuse loss (e.g., which may be caused by non-neural factors such as cornea/lens opacity), in order to better visualize focal loss that is the hallmark of eye disease, such as glaucoma.

Visual function in AMD is measured using perimetry, and often a specific variant of perimetry called microperimetry, where a denser test grid is used to examine function in the central visual field and is often used in conjunction with eye-tracking. This typically measures contrast thresholds for stimuli of constant area (0.43 deg. diameter) and duration (200 ms) at various preselected locations within the central 10° of the visual field.

Whilst perimetry and microperimetry are widely used to assess visual function in healthy individuals and in conditions such as glaucoma and AMD, they suffer from a lack of sensitivity to subtle changes in visual function, high test-retest variability, and the inability to measure functional loss in advanced disease due to a limited dynamic range.

Spatial summation is the term given to the ability of the visual system to combine and sum light energy across space. This can be seen in the response to a visual stimulus, in that light energy spread across a stimulus, or multiple stimuli in sufficiently close proximity, can be summed (completely or partially) to initiate a single signal response. For example, if certain conditions are favourable, the energy of two adequately small but identical stimuli of a given brightness, presented in close proximity within the receptive field of a cell, would be summed, to give the perception of a single stimulus. For a range of small stimuli, there is complete summation of light energy within the stimulus at threshold (complete spatial summation). Within this range, stimulus area and intensity are inversely proportional at threshold (i.e. area×intensity=k). This is known as Ricco's Law. The largest stimulus area for which Ricco's law holds is known as Ricco's area, or the area of complete spatial summation, or the critical area. Spatial summation curves can be determined by measuring sensitivity for a range of stimuli of different areas.

Temporal summation is similar to spatial summation in that the visual system can combine and sum light energy over time. For stimuli of sufficiently short duration, all light energy within the stimulus is summed (complete temporal summation), such that at threshold, stimulus duration and intensity are inversely related (duration×intensity=k). This is known as Bloch's law, and the longest stimulus duration for which this law holds is called the critical duration. In a similar manner to spatial summation, two discrete stimuli presented in succession with a sufficiently short interval will also be summed and perceived as being a single stimulus. When the maximum inter-stimulus interval is exceeded, successive stimuli will however be perceived as separate. Temporal summation curves can be determined in the same way as spatial summation curves, by measuring sensitivity/threshold for a range of stimuli of different durations rather than areas.

With the above in mind, we have developed a new form of perimetry for measuring eye and visual pathway function, including disease associated therewith, which, advantageously can tap into changes in spatial and/or temporal summation, ideally but not exclusively Ricco's area and/or the critical duration, associated with the function of that pathway or representative of said diseases. Moreover, this perimetry, which can be undertaken manually or in an automated fashion, provides more sensitive results than standard perimetry, thus enabling early changes in vision to be identified and so facilitating preventative or remedial action, where appropriate.

### SUMMARY

According to a first aspect of the invention, there is provided a method for measuring visual field sensitivity comprising:
i) presenting to a subject a fixed-luminance stimulus of a first size at one, or more, location(s) in the subject's visual field and recording the response of the subject to the said stimulus at each one or more locations in the visual field;
ii) presenting to the same subject a fixed-luminance stimulus of a second size at the same one, or more, location(s) in the subject's visual field and recording the response of the subject to the said stimulus at each one, or more, location(s) in the visual field;
iii) optionally, repeating step ii) using one or more further fixed-luminance stimuli of one or more further sizes;
iv) using the subject's responses in parts i)-iii) to determine a threshold for the detection of said fixed-luminance stimuli of different sizes (or areas) at said one or more locations;
v) comparing the determined threshold of part iv) for said subject with either, or both, the threshold in an age-matched normal group or the threshold(s) in at least one prior determined baseline to identify any change in visual field sensitivity; and, optionally,
vi) where a change in visual field sensitivity is found, using it as an indicator of a change in visual pathway function or disease.

Reference herein to a fixed-luminance stimulus is to a substantially fixed-luminance stimulus that is fixed or varies only by a minor amount so as to appear the same or fixed.

Reference herein to a baseline is to a prior measurement of the subject's visual field sensitivity when a prior test was performed, ideally but not exclusively, the test of the invention but any other form of perimetry may be used in part v) of the claimed method to establish said baseline, preferably, where a different method has been used to establish the baseline, the data are converted, as herein described, to be compatible with the data obtained using the method of the invention. In the alternative, reference herein to a baseline is to a prior measurement of a selected subject's visual field sensitivity when a prior test was performed, ideally but not exclusively, the test of the invention but any other form of perimetry may be used in part v) of the claimed method to establish said baseline, preferably, where a different method has been used to establish the baseline, the data are converted, as herein described, to be compatible with the data obtained using the method of the invention.

Notably, in some clinical cases, multiple baselines may be used to assess visual field sensitivity. In other words, multiple baselines may be used in the determination of a change in sensitivity. For example, if a subject underwent 3 tests in short succession, these could, independently or collectively, be considered (a) baseline(s). Then, if the subject were to undergo a further follow-up test after a period of time, and their vision differed sufficiently from the baseline(s), the clinician might have greater confidence that any change is a true change.

In fact, baseline determination could be done retrospectively and/or prospectively, e.g.,
a. One or more tests are conducted prior to a subject having a suspected eye/visual pathway disease (e.g., as part of routine care).
b. One or more tests are conducted when eye/visual pathway disease is suspected or confirmed, and then the subject undergoes follow-up with subsequent tests to determine if there is any change from the initial or previous test(s).

In the case of a. above, the current visual field test result is compared with one or multiple baseline(s) (i.e., one or multiple tests that were carried out previously) to determine if the most recent result is sufficiently different from that subject's previous normal result(s), taking into account their own normal variability, where available.

In the case of b. above, one or multiple baseline tests are undertaken, the subject is followed-up with subsequent tests (e.g., after a few months), and the most recent follow-up results are compared with the one or multiple baseline(s) to see if the most recent result is sufficiently different from the one or more multiple baseline(s), taking into account their own normal variability and the between-test variability in the baseline tests.

Broadly speaking, there are two types of ‘analysis’ that one would use (either computationally or intuitively) to determine if there has been any change:
i) Event-based analysis: this is where one compares the most recent test result with a baseline result, and if it falls outside expected test-retest variability limits for the baseline result, change is likely to have occurred. For example, if the baseline sensitivity were 30 dB and we know that normal test-retest variability limits for that sensitivity level are (30−x) dB to (30+x) dB where x is a positive number, then if today's result is (30−x−y) dB, where y is a positive number, the result falls outside that range and therefore is likely to be due to true change. In some cases, ‘baseline’ can be an average of two or more previous tests.
ii) Trend-based analysis: this is where one considers the direction and rate of change over time and determines if it is positive, negative, or flat, as well as the magnitude and significance (or otherwise) of any rate of change. A simple example of such a method is linear regression of sensitivity over time. Clinicians would either do this computationally, or intuitively (e.g., considering how the values change over time, and then coming to a clinical judgement about whether any observable change over time is notable or not).

Reference herein to a threshold determined by said area-modulated stimuli is a threshold above which said stimuli is substantially detected and below which said stimuli is substantially not detected.

Those skilled in the art of perimetry will appreciate, the stimulus is used to stimulate vision, and we manipulate its size, or area, in the test to determine a certain threshold. Threshold is typically the smallest spot that is visible, or it can be a value that is scaled to the smallest spot that is visible.

Reference herein to a threshold that is scaled to the smallest spot that is visible means a threshold where a test stimulus spot is so small it is seen only x % of the times it is presented, where x is typically 50% but it may be higher, e.g., 79% where patient fatigue or test comfort is a consideration for the subject/patient. This simply means that rather than it being the smallest stimulus that is visible, it is slightly bigger than the smallest stimulus that is visible but scaled to it. So, if the 50% seen value were to change with disease, so too would the 79% value.

In our test, we use the stimuli to find threshold, where threshold is at or smaller than Ricco's area, as shown in the schematic in FIG. 9. When working the method, different conventional mathematical formulae can be used to decide on the area size of each next stimulus to use. When testing, we use stimuli that are larger and smaller than a predicted threshold in order to measure where threshold is. (A form of bracketing, to find what values of stimulus can and can't be seen). Essentially, we want to find the stimulus area that matches or is scaled to Ricco's area.

Accordingly, in yet a further preferred method of the invention the size of the area stimulus at threshold is equal to or smaller than Ricco's area.

In a preferred embodiment of the invention step v) includes statistical analysis to determine the probability that a deviation at a given location, or a number of locations, or the sum of a number of locations is due to chance or is representative of disease.

In yet a further preferred method of the invention said luminance of said stimulus is fixed at or greater than the luminance of a stimulus equivalent to Ricco's area at luminance threshold for that individual in a previous test or for age-similar healthy individuals.

In yet a further preferred method of the invention the said stimulus at each location is presented for a fixed duration, ideally but not exclusively, that is at or (scaled to be) shorter than the critical duration for age-similar healthy individuals.

In yet an alternative method of the invention said stimulus duration is modulated so that the stimulus of part i) of a first fixed size is presented for a fixed first duration but the stimulus of part ii) of a second fixed size is presented for a fixed first duration or a fixed second different duration, more preferably still, where the option of part iii) is used and so step ii) is repeated using one or more further fixed-luminance stimulus of one or more further sizes, the said stimulus duration may be of any first, second or further fixed duration. In this way the test can be used to examine temporal summation, ideally but not exclusively the critical duration.

In yet a further preferred method of the invention the said stimuli are presented at different locations, ideally in close proximity, to test spatial summation, ideally but not exclusively Ricco's area.

In yet an alternative method of the invention said stimuli are presented at different locations so that the test i)-vi) of the invention is undertaken at different locations across the visual field. In some instances, the test i)-vi) is undertaken at a first chosen location to determine the visual field threshold at that location before the test i)-vi) is repeated at another location. More typically, testing to determine threshold is undertaken at a number of locations in an interwoven manner so that, e.g., a number of stimuli of part i) are presented at a number of locations and the subject's/patient's response is recorded and then part ii)/iii) of the test is undertaken at the same selected number of locations or, even different locations, and the subject's/patient's response is recorded. This is repeated until a number of locations have received a sufficient number of stimuli of different sizes to determine a threshold at each of those locations. Indeed, in suprathreshold testing, a subject/patient is repeatedly presented with a number of area-modulated stimuli of fixed luminescence across the visual field, either simultaneously or sequentially, where the stimuli are expected to be detectable by the subject based on baseline(s) or a typical healthy subject of the same age and demographic, and the response at each location to each different sized stimulus is repeatedly recorded to determine if threshold has changed sufficiently at any location such that it surpasses the stimulus area expected to be detectable. Such a change would indicate a change in visual field sensitivity, and therefore an indicator of a change in visual pathway function or disease (as per part vi) of the claimed test).

In a preferred method of the invention, the stimulus of part i) of a first fixed size is presented at a first location and a second stimulus of the same size (or a different size) is presented at a second location, ideally in close proximity to said first location. In this way the test can be used to examine spatial summation, ideally but not exclusively Ricco's area.

Without wishing to be bound by theory, we consider the cornerstone of any test of visual field sensitivity is the stimulus. This is because when neurons or neural networks are damaged, their ability (or the ability of remaining healthy neurons) to process basic configurations (e.g., light vs dark, colours, edges, specific patterns, etc.) is altered. Thus, if a stimulus is configured in a specific way such that the altered network cannot process it correctly, this will manifest as a functional anomaly. In order to determine the extent of change or damage, the stimulus must vary in one or more of its features, and the aim is typically to determine the best signal to uncover this change or damage and so the limits of the ability of the neural system to respond. The magnitude of the sensitivity of the neural network to the stimulus is known as the ‘disease signal’. However, variability in the probing effectiveness of a test stimulus can arise from multiple factors, including those that are independent of the test (e.g., internal neural fluctuations, co-morbidities, fatigue or motivation of the individual being tested), and those that are due to, or altered by the test (e.g., the thresholding algorithm and response paradigm). In order to compare the utility of stimuli directly, it is necessary to remove all possible confounders that might bias the measurement or the interpretation of findings. With this in mind, we have discovered that presentation of fixed-luminance, area-modulated and, ideally but not exclusively, fixed-duration stimuli is an extremely effective way of measuring visual field sensitivity.

In a preferred method of the invention said stimulus is a spot shaped or circular stimulus.

In yet a further preferred method of the invention, said disease is one that affects spatial and/or temporal summation of the visual field, ideally but not exclusively Ricco's area and/or the critical duration, such as glaucoma or age-related macular degeneration (AMD).

In yet a further preferred embodiment of the invention the method comprises converting the stimulus into an energy value using the following formula:

$$\begin{matrix}E = A \times D \times L \\ E = A \times D \times ML \times {10}^{( \frac{- dB}{10} )}\end{matrix}$$

Where A=stimulus area (ideally, but not exclusively in deg2), D=presentation duration (ideally, but not exclusively in seconds), and L=stimulus luminance (ideally, but not exclusively in cd/m2). Stimulus luminance can be calculated from the dB value associated with the HFA using the following equation (eq 3):

$$L = ML \times {10}^{( \frac{- dB}{10} )}$$

Where ML=maximum luminance in cd/m2 (ideally, but not exclusively in cd/m2=asb/π), thus combining the afore two equations to convert from dB to E (eq 4):

$$E = A \times D \times ML \times {10}^{( \frac{- dB}{10} )}$$

In yet a further preferred method of the invention, the method involves converting the baseline data obtained using a prior measurement of the, or a, subject's visual field sensitivity into a stimulus energy value (equivalent to those used in the test of the invention), using the following formula, before step v) is performed

$${E}_{AMP} = {S}_{1} \times [ {E}_{SAP} - k ] \times [ {E}_{SAP} < k ) ] + \{ {E}_{SAP} + {I}_{2} ]$$

where E_{SAP} is SAP sensitivity converted to energy using Equations 3 and 4 of claim 10, E_{AMP} is the equivalent energy threshold for AMP, S₁ is the slope of the first segment in the two-phase model, I₂ is the intercept of the second segment in the two-phase model, and k is the energy value for a stimulus of equivalent area to Ricco's area and the luminance level of this area at threshold.

Alternatively, the method of the invention involves converting the baseline data obtained using a prior measurement of the, or a, subject's visual field sensitivity into a stimulus energy value (equivalent to those used in the test of the invention), using the following formula, before step iv) is performed

$${E}_{AMP} = {S}_{1} \times [ {E}_{SAP} - ( k * a ) ] \times [ {E}_{SAP} < ( k * a ) ] + [ {E}_{SAP} + {I}_{2} ]$$

where E_{SAP} is SAP sensitivity converted to energy using Equations 3 and 4, E_{AMP} is the equivalent energy threshold for AMP, S₁ is the slope of the first segment in the two-phase model, I₂ is the intercept of the second segment in the two-phase model, and k is the energy value for a stimulus of equivalent area to Ricco's area and the luminance level of this area at threshold, and we introduce a parameter a, to adjust the contrast level for the stimulus equivalent in area to Ricco's area.

According to a second aspect of the invention, there is provided an instrument for measuring visual field sensitivity comprising:
i) at least one light stimulus emitter adapted to emit fixed-luminance stimuli of variable sizes for stimulating, at least, a first location in a subject's visual field, wherein said light stimulus emitter is moveable relative to the subject's visual field, whereby a further fixed-luminance light stimulus of a first or different size can be emitted to stimulate said first or a different location in the same subject's visual field;
ii) a recording device for recording the subject's response to said fixed-luminance light stimulus at said first or different location in the subject's visual field;
iii) a processor for capturing the subject's response to said light stimuli at each, or a selected one or more, location(s) in said subject's visual field and adjusting said light stimulus emitter to emit a further fixed-luminance stimulus of a different size or targeted to a different location and then using the subject's responses to determine a threshold for the detection of said area-modulated stimuli at said one or more locations;
iv) the or a further processor for comparing the threshold of iii) with either, or both, the threshold in an age-matched normal group or the threshold in a prior determined baseline to identify a change in visual field sensitivity and where a change in visual field sensitivity is found, using it as an indicator of a change in visual pathway function or disease.
v) a display device for displaying the results of step iii) and/or iv) to a user.

Reference herein to at least one light stimulus emitter that is moveable relative to the subject's visual field, is reference to at least one light stimulus emitter that moves with respect to the subject's visual field, or reference to multiple light stimulus emitters each one or more emitting fixed-luminance stimuli sequentially or concurrently and arranged to target different locations in a subject's visual field.

In a preferred embodiment of the invention said light stimulus emitter is a screen adapted to emit a beam of light within the area ranging from a fraction—a full screen, including all possible pixel increments therebetween.

In a preferred embodiment of the invention step iv) includes statistical analysis to determine the probability that a deviation at a given location, or a number of locations, is due to chance or is representative of disease.

In yet a further preferred method of the invention said luminance of said stimulus is fixed at or greater than the luminance of a stimulus equivalent in size to Ricco's area at luminance threshold for age-similar healthy individuals, ideally at an equivalent visual field location.

In yet a further preferred method the said stimulus at each location is presented for a fixed duration, ideally, that is at, shorter than, or shorter than and scaled to the critical duration for age-similar healthy individuals at an equivalent visual field location. Typically, a short duration signal, certainly one less than the critical duration, is desirable because it has higher probing potential, meaning it uncovers changes in visual field sensitivity.

In yet an alternative embodiment of the invention said stimulus duration is modulated so that the stimulus of part i) of a first fixed size is emitted for a fixed first duration but the stimulus of part i) of a second fixed size is emitted for a fixed first or second different duration.

More preferably still, where a further fixed-luminance light stimulus of a different size is presented to said first or further, different, locations in the same subject's visual field, the duration of one or more fixed-luminance light stimulus is modulated so that it differs as different area stimuli are used and/or between locations on the subject's visual field.

In yet a further preferred embodiment of the invention the size of the area of the stimulus is at or smaller than Ricco's area at luminance threshold and, additionally or alternatively, the duration of the stimulus is at or shorter than the critical duration at threshold.

In the claims which follow and in the preceding description of the invention, except where the context requires otherwise due to express language or necessary implication, the word “comprise”, or variations such as “comprises” or “comprising” is used in an inclusive sense i.e., to specify the presence of the stated features but not to preclude the presence or addition of further features in various embodiments of the invention.

All references, including any patent or patent application, cited in this specification are hereby incorporated by reference. No admission is made that any reference constitutes prior art. Further, no admission is made that any of the prior art constitutes part of the common general knowledge in the art.

Preferred features of each aspect of the invention may be as described in connection with any of the other aspects.

Other features of the present invention will become apparent from the following examples. Generally speaking, the invention extends to any novel one, or any novel combination, of the features disclosed in this specification (including the accompanying claims and drawings). Thus, features, integers, characteristics, compounds or chemical moieties described in conjunction with a particular aspect, embodiment or example of the invention are to be understood to be applicable to any other aspect, embodiment or example described herein, unless incompatible therewith.

Moreover, unless stated otherwise, any feature disclosed herein may be replaced by an alternative feature serving the same or a similar purpose.

Throughout the description and claims of this specification, the singular encompasses the plural unless the context otherwise requires. In particular, where the indefinite article is used, the specification is to be understood as contemplating plurality as well as singularity, unless the context requires otherwise.

### BRIEF DESCRIPTION OF THE DRAWINGS

An example according to the present invention will now be described with reference to the following methods and accompanying Figures, in which:

FIG. 1 is a Schematic of stimulus configurations (ref: Rountree L, Mulholland P J, Anderson R S, Garway-Heath D F, Morgan J E, Redmond T. Optimising the glaucoma signal/noise ratio by mapping changes in spatial summation with area-modulated perimetric stimuli. Sci Rep 2018; 8:2172). ‘A’ refers to area-modulated stimuli. ‘GIII’ refers to Goldmann III (control) used in conventional perimetry. Length of arrows (difference between curves measured along axis of modulation) denotes the hypothesized ‘disease signal’. Right schematic demonstrates how stimuli vary with patient responses during the test (e.g., ‘GIII’ varies in luminance with area and duration fixed, ‘A’ varies in area with luminance fixed and fixed or varying duration).

FIG. 2 plots the vertical distance between temporal summation curves for glaucoma patients and controls for a conventional Goldmann III stimulus (black) and a Ricco's area-scaled stimulus (red). Conventional stimulus duration (200 ms dotted line) is shown for reference. Larger values on the y-axis (vertical) denote greater disease signal. Label A shows the disease signal (difference in threshold between patients and controls) for a conventional Goldmann III stimulus with 200 ms duration. Label B shows disease signal for a Goldmann III stimulus at the average critical duration in healthy observers. Disease signal for stimuli of shorter duration than the critical duration can be predicted from the black line for x-values lower than the critical duration. Labels C and D show the same values for a Ricco's area-scaled stimulus. Disease signal for a stimulus within Ricco's area and the critical duration can be predicted from the red line for x-values lower than the critical duration D.

FIG. 3 Disease signal expressed as difference in contrast energy thresholds for each stimulus form in the superior hemifield. Included for reference are individual data points (blue spots), zero test sensitivity line (dashed line) and statistical significance markers for post-hoc Wilcoxon signed-rank tests, after Holm-Bonferroni correction.

FIG. 4A-C, E-G: Simulated shifts in Ricco's area along the area axis. D (blue shading): Data showing the actual difference in Ricco's area found in that study. H: Disease signal representing area-modulated stimuli (distance between curves on the x-axis within Ricco's area) plotted against disease signal representing conventional luminance-modulated GIII stimuli. A green segmented regression line is fitted to the simulated data. I: Thresholds measured with area-modulated stimuli plotted against thresholds measured with GIII stimuli. Data are fitted with segmented regression. The fitted model shows close agreement with the model determined from simulated data.

FIG. 5 shows disease signal for area-modulated stimuli (A) AMP and conventional SAP Goldmann III stimuli (GIII). A value of 0 on the y-axis represents ‘no discriminability’ between glaucomatous and normal vision.

FIG. 6 shows Boxplots that show RA estimates at 2.5° and 5° visual field eccentricity for healthy controls (blue) compared to AMD participants (red). Individual data points for individual observers are included for reference.

FIG. 7 shows Critical duration estimates at 2.5° (a) and 5° (b) visual field eccentricity for healthy controls (blue) and participants with AMD (red) with a GIII stimulus and Ricco's area-scaled stimuli. Outliers are represented by ‘+’ markers.

FIG. 8 shows Mean disease signal for all six stimulus durations with the GIII stimulus (blue) compared to Ricco's area-scaled stimuli (purple) at 2.5° (a) and 5° (b) eccentricity.

FIG. 9 schematically shows use of an area-modulated stimulus, Y axis, during a typical testing period, x axis, to stimulate vision. Different size stimuli (dots) are presented during the course of the test, those above the threshold are visible and so register as seen those below are not visible to the subject and so register as unseen. Eventually as more dots of different sizes are presented a threshold is determined. Threshold is typically the smallest spot that is visible, or it can be a set value that is scaled to the smallest spot that is visible. In our test, threshold is at or smaller than Ricco's area, as shown in this figure. Notably, the order or pattern of stimulus presentation may vary according to each test, similarly, the mathematical formula for deciding each test value my vary depending upon the algorithm used to effect the perimetry test.

FIGS. 10-13 shows test results obtained using 17 patients when measuring visual field function using the method of the invention and using the Goldmann methods as comparators. Graphs 10-13, A-D, show threshold (limit of vision) for healthy controls (black/dark dots) and glaucoma patients (blue/light dots). The regression line is plotted through the control data only and describes how the limits change as a function of age (x axis). The way the y-axis works is that higher numbers=worse vision. So, glaucoma patients' data should be higher than what would be predicted for someone healthy of their age, i.e., the further the blue/light dots are away from the black line (in a vertical direction) the worse their visual field sensitivity. Also in these FIGS. 10-13, 9.9° refers to a location that is 9.9 degrees of visual angle away from central fixation, 13° refers to a location that is 13 degrees of visual angle away from central fixation, 16° refers to a location that is 16 degrees of visual angle away from central fixation, and 20° refers to a location that is 20 degrees of visual angle away from fixation.

FIG. 10 shows data for a single stimulus type: fixed luminance, variable area, fixed duration at 16 ms and within the critical duration (we call this AMP 16 ms—area-modulated perimetry stimulus @ 16 msec);

FIG. 11 shows data for a single stimulus type: fixed luminance, variable area, fixed duration near 200 ms and outside the critical duration (we call this AMP 200 ms—area-modulated stimulus @ 200 msec);

FIG. 12 shows data for a single stimulus type: fixed area (Goldmann III or “GIII”), variable luminance, fixed duration near 200 ms and outside the critical duration (this is the current clinical reference standard); and

FIG. 13 shows data for a single stimulus type: fixed area (Goldmann V or “GV”), variable luminance, fixed duration near 200 ms and outside the critical duration.

FIGS. 14-17 takes the data shown in FIGS. 10-13 and shows how much each glaucoma patient's threshold differs from the predicted value from their age (i.e., “difference from an age-matched control” or “disease signal”). In these FIG. 14-17, if data are clumped around the horizontal line, threshold is much the same as that of an age-matched control; if they are above the line, threshold is higher (i.e., vision is worse) than an age-matched control; if they are below the line, threshold is lower (i.e., vision is better) than an age-matched control.

FIGS. 14-17 show this analysis for each of the test locations and for each of the 4 stimulus types as described above in FIGS. 10-13. It can be seen that data obtained using the conventional Goldmann methods of testing (FIGS. 16 & 17) approximates more closely to the control age-matched horizontal line, thus being less able to distinguish early stages of disease. Whereas, in contrast, data obtained using the methods of the invention, AMP perimetry, are further from the horizontal line (higher), thus being able to distinguish early stages of disease. (Data includes the median disease signal for each stimulus and each location).

FIG. 18 shows the pooled data in FIGS. 11-13 presented as boxplots. These boxplots show disease signal for each of the stimulus types and for each visual field location separately. Disease signal in FIGS. 14-17 is the distance between the points and the horizontal lines in the plots. In other words, it is how much each individual location/patient differs from what would be predicted for an age-similar normal test. Clearly the higher signals being worse than an age-matched control and so representing the possibility (at least) of disease. This is a standard method of measuring the magnitude of disease with a particular stimulus in the clinical setting. In FIG. 18, the median disease signal for each stimulus is shown as a horizontal black line and it can be seen that this line is higher for the area-modulated stimuli (AMP) than for the luminance-modulated stimuli (GIII or GV), indicating AMP will be more sensitive/discriminatory for determining a disease signal. Further, FIG. 18 also shows that AMP16 and AMP200 (i.e., area modulated stimuli) at all individual locations show a greater disease signal (i.e., greater vertical distance from the median disease signal, indicating with greater reliability the possibility of disease) than the standard GIII & GV at all test locations.

### DETAILED DESCRIPTION

### Methodology

### Area-Modulated Stimuli

### Proof-of-Concept for Area-Modulated Stimuli

Using glaucoma as a test eye disease, the method of the invention was undertaken to see if it had the necessary forensic ability to uncover early loss of visual sensitivity.

The method of the invention was practised using four different stimulus types on five separate occasions, over five visits, within 11 weeks and the data obtained were used to generate conventional spatial and temporal summation curves.

### Spatial Summation

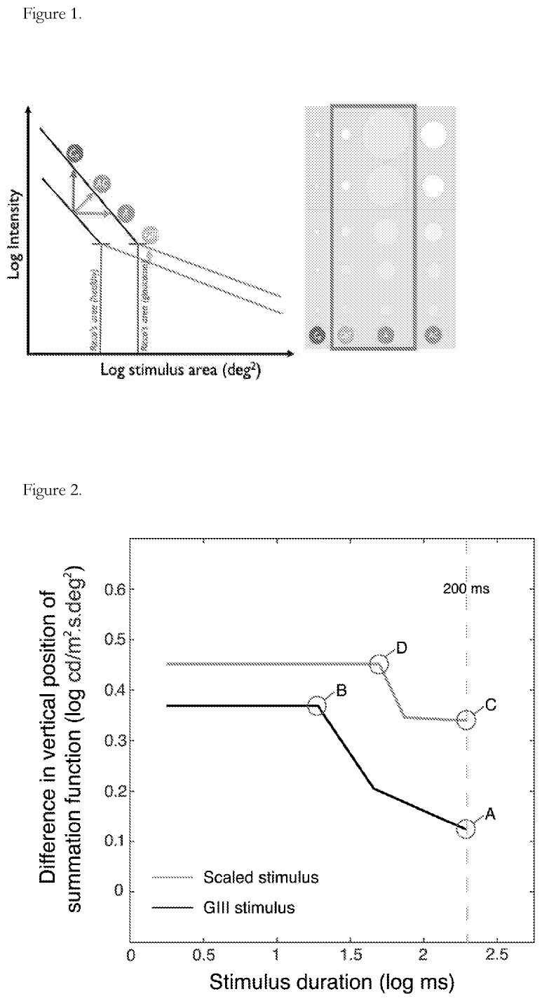

The threshold to conventional fixed-area, fixed-duration, luminance-modulated stimuli is represented on both the spatial and temporal summation curves shown in FIG. 1. In plotting full spatial summation curves for patients with glaucoma and healthy controls, we determined the position of the greatest separation between the curves. The distance between summation curves at a given point is the disease signal as it represents the difference in sensitivity/threshold between patients and controls.

FIG. 1 shows the different prognostic effect various parameters have on measuring visual sensitivity, where A refers to area-modulated stimuli, ‘GIII’ refers to Goldmann III (used as a control) that is used in conventional perimetry. The length of the arrows is representative of the difference between curves when measured along the axis of modulation, this length denotes the size of the ‘disease signal’. The larger the arrow the more prognostic the method. It can be seen that arrow A is much larger than arrow GIII, the current gold standard.

Moreover, if we compare these two parameters, in terms of how stimuli vary with patient responses during the test (FIG. 1 Right schematic) we show GIII varies in luminance with the area fixed, whereas A varies in area with the luminance fixed.

FIG. 1 also shows a greater disease signal, when measured along the area axis within Ricco's area for a healthy individual—an individual with glaucoma i.e., within the sensitive region for identifying this disease or changes in this disease. This greater disease signal is obtained when varying (modulating) the stimulus along the same vector in a visual field test. In this case, the disease signal for stimuli of fixed intensity, varying in area only (A in FIG. 1) was greater than that for stimuli with fixed area, varying in intensity only (GIII in FIG. 1) in a perimetry test.

### Temporal Summation Curves

On plotting the temporal summation curves for glaucoma vs healthy individuals, it was observed that they are very close at the 200 ms point, FIG. 2, this is the duration value used herein along with the Goldmann III stimulus in Standard Automated Perimetry. We infer that disease signal is greater still for stimuli shorter than the critical duration. Furthermore, it can also be inferred that stimuli varying in duration within a test also enable a greater disease signal.

A recent (unpublished) study by our group demonstrated statistically significant improvements in disease signal with a reduced-duration stimulus, compared with the same stimulus of a longer duration, confirming the prediction that stimuli shorter than Bloch's duration would bring superior performance.

We hypothesised that the likely reason for this may be linked to recent findings of pre-morbid dysfunction (‘sickness’) in retinal ganglion cells. In vitro experimental glaucoma, as well as studies of post-mortem human eyes, have pointed to early disturbances in retinal ganglion cells that may precede cell death, and there is some suggestion that any effect on vision may even be reversible if pre-morbid dysfunction is detected sufficiently early. The notion of detecting pre-morbid dysfunction with perimetry has not received much attention, mainly because of the high variability in measuring visual field sensitivity with perimetry, i.e., such subtle changes would not be detectable in the presence of such high variability. However, with stimuli that bring the benefit of reduced variability, greater disease signal, and higher signal to noise (SNR) it is theoretically possible to measure much subtler changes than at present.

To explain our findings of a greater disease signal with reduced-duration stimuli, we propose the following. In a given region of healthy retina with many retinal ganglion cells, each has the capability of relaying small signals. If, in glaucoma, cells undergo a period of sickness prior to death, then those sick cells should only be capable of relaying strong signals that are easy to see (e.g., long duration). Thinking about this from the opposite end, if a stimulus is much more difficult to see (e.g., a shorter duration) and the cell requires the maximum metabolic demand to be able to relay the information, then theoretically, only the healthiest cells will relay the signal. The dead and dysfunctional cells will not. Thus, it is possible that a short-duration stimulus can improve the performance of a stimulus, by exploiting the vulnerabilities of dysfunctional cells prior to cell death. Regardless of the cause, this can still be considered ‘reduced visual field sensitivity’, given the significantly greater disease signal.

### Data Integration

One of the major challenges for new perimetry is the ability to incorporate legacy data for continuity of patient care. As mentioned above, many measurements are required, often over many years, to identify glaucomatous visual loss and/or progression. Typically, new perimetry tests employ stimuli that are so different in configuration from those in conventional perimetry that their units are not comparable, and therefore the ability to identify true visual change can be compromised. For example, sensitivity measured with a grating stimulus is not directly comparable to sensitivity measured with a spot stimulus and likely taps into a different visual processing stream. By extension, units of sensitivity differ greatly between tests, even though they often appear to be comparable. For example, the unit of sensitivity in conventional perimetry is the decibel (dB). In the Humphrey Field Analyzer (HFA) this is defined by the following equation (eq 1):

dB=10×log₁₀(μ_{ML})
where μML is the attenuation from the maximum possible luminance.

If sensitivity is 20 dB, this means that the stimulus luminance at threshold/sensitivity was 2 log units lower than the maximum luminance deliverable by the hardware. In the HFA, the maximum luminance is 10,000 asb (3,183 cd/m2).

Other instruments and tests also employ the dB as the unit of sensitivity, but the definition of the dB can vary (e.g., in Frequency Doubling Matrix perimetry [FDT], the dB is defined as dB=20×log₁₀ (μ_{ML}).

Furthermore, the maximum luminance of alternative instruments is usually different to that of the HFA. Therefore, 20 dB measured on one instrument is not the same sensitivity as 20 dB measured on another instrument, nor is 20 dB measured with one stimulus form the same as 20 dB measured with another stimulus form. Thus, even though the dB is apparently the same between instruments (leading many studies to compare measurements in dB as though they were the same unit), they are not directly comparable. Importantly, in the identification of glaucoma progression, if a patient's sensitivity is reduced from (for example) 28 dB to 23 dB over a period of time when measured on the same instrument, clinicians can be relatively confident that this represents true deterioration than if the former were measured on a legacy instrument and the latter measured on a newer test instrument with a different definition for the dB unit. This dilemma, along with other factors such as the learning effect that accompanies new tests with vastly different stimuli or tasks, can cause caution among clinicians who understandably see the introduction of new perimetry as potential disruption to clinical care.

However, the method of the invention, termed herein Area-Modulated Perimetry (AMP) offers a solution to this problem in two ways.

Firstly, regardless of test modulation, area-modulated stimuli are spot stimuli with a luminance that is greater than the background. Thus, the task of the patient is unaltered from the conventional test, meaning there is no or minimal requirement to re-learn.

Secondly, and importantly, because area-modulated stimuli are spot stimuli, like conventional luminance-modulated stimuli, measurements with either stimulus can be converted to the other using a two-step process, as described below.

### Step 1: Conversion to a Common Measurement Scale

Both stimulus forms can be represented on a common energy (E) scale using the following equation (eq 2):

$$E = A \times D \times L$$

Where A=stimulus area (ideally but not exclusively in deg2), D=presentation duration (ideally but not exclusively in seconds), and L=stimulus luminance (ideally but not exclusively in cd/m2). Stimulus luminance can be calculated from the dB value associated with the HFA using the following equation (eq 3):

$$L = ML \times {10}^{( \frac{- dB}{10} )}$$

Where ML=maximum luminance ideally but not exclusively in cd/m2 (cd/m2=asb/π).

Combining the afore two equations to convert from dB to E (eq 4):

$$E = A \times D \times ML \times 1 {0}^{( \frac{- dB}{10} )}$$

### Step 2: Conversion Between Methods

Conversion between energy values for Standard Automated Perimetry (SAP) and AMP requires a consideration of the relative area of the conventional Goldmann III stimulus and Ricco's area. With an enlargement of Ricco's area in glaucoma and other diseases that affect spatial summation, the relationship between thresholds measured with SAP and AMP is non-linear. Therefore, a non-linear function is required to convert legacy values to those compatible with AMP.

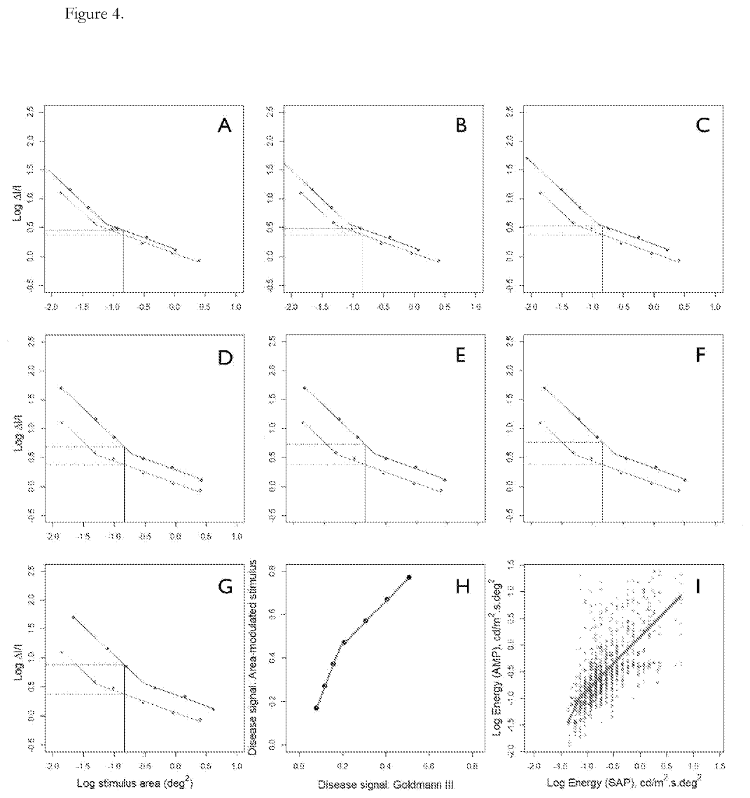

We undertook re-analysis and re-interpretation of prior-acquired data using SAP. FIG. 4(D) shows spatial summation data for glaucoma patient (blue line) and healthy control (green line) cohorts, replotted as a reference. The distance between any point on the control curve to any point on the glaucoma curve is a measure of disease signal for a stimulus that varies in area and/or luminance along that specific vector. For example, for a conventional Goldmann III stimulus, varying in luminance, disease signal is the vertical distance between the two curves for a stimulus area of −0.838 log deg² (the area of a Goldmann III stimulus). Disease signal for area-modulated stimuli is represented by the horizontal distance between the two curves for a fixed luminance or fixed contrast stimulus (e.g. at Ricco's area).

Changes in disease signal for an area-modulated stimulus was plotted as a function of the change in disease signal for a Goldmann III stimulus, with simulated advancing depth of visual field damage. FIG. 4 visually demonstrates the change in contrast threshold for a Goldmann III stimulus for a stimulated horizontal displacement of the spatial summation curve in glaucoma. Seven possible separations of the glaucoma and healthy spatial summation curves are shown in (FIG. 4 (A-G)).

Disease signals were calculated for AMP and GIII stimuli for seven evenly-spaced displacements of the spatial summation curve in glaucoma; those for AMP were plotted as a function of those for the GIII stimulus FIG. 4(H). A two-phase regression model was fitted to the data using an iterative fitting technique. When Ricco's area exceeds the area of the Goldmann III stimulus, threshold is determined by complete spatial summation as is governed by Ricco's law. Thresholds measured with area-modulation stimuli are also determined by complete spatial summation. Therefore, when Ricco's area exceeds the area of the Goldmann III, the relationship between area-modulated stimulus and Goldmann III energy thresholds will be linear. The slope of the second segment of the two-phase regression model was therefore constrained to a value of 1. The parameters for the intercept of the second segment, the slope of the first segment, and the breakpoint between segments were allowed to vary during the fitting procedure. The breakpoint value represents the disease signal for the area-modulated stimulus when Ricco's area matches the area of a Goldmann III stimulus, and the point at which the relationship between AMP and Goldmann III energy thresholds changes.

Next, unpublished data from a cohort of 15 patients with glaucoma, pooled across 18 test locations and 5 visits each were used to test the accuracy of the conversion data.

SAP sensitivity was measured with Goldmann III stimuli (SAP; HFA, Carl Zeiss Meditec, Dublin, CA) and converted to energy values using the equation (eq 4):

$$E = A \times D \times ML \times 1 {0}^{( \frac{- dB}{10} )}$$

Energy thresholds were also measured in the same locations with area-modulated stimuli, driven by a staircase thresholding algorithm.

Energy values for area-modulated stimuli were plotted against those for SAP and iterative two-phase segmented regression was performed on the data. The parameters for the free variables that were determined by the fitting procedure were used as starting values for an iterative two-phase segmented regression applied to the patient data.

FIG. 4(I) shows the model from FIG. 4(H) superimposed on the patient data (green segmented line). The breakpoint was fixed at the energy value on the x-axis for a stimulus with the same area as Ricco's area and a luminance level of this area at threshold. The iterative two-phase regression analysis on the patient data found values for the free parameters that were almost identical to those determined from the theoretical model (FIG. 4(I), red segmented line).

The relationship between energy values for the two methods can be accurately described by the following equation (eq 5):

$${E}_{AMP} = {S}_{1} \times [ {E}_{SAP} - k ] \times [ {E}_{SAP} < k ) ] + [ {E}_{SAP} + {I}_{2} ]$$

Where E_{SAP} is SAP sensitivity converted to energy using Equations 3 and 4. E_{AMP} is the equivalent energy threshold for AMP, S₁ is the slope of the first segment in the two-phase model, I₂ is the intercept of the second segment in the two-phase model, and k is the energy value for a stimulus of equivalent area to Ricco's area and a luminance level of this area at threshold, scaled for background adaptation level, as appropriate.

It has been shown that although Ricco's area does not change with age, contrast thresholds are uniformly higher with increasing age. Therefore, we introduce a parameter a, to adjust the contrast level for the stimulus equivalent in area to Ricco's area (eq.6):

$${E}_{AMP} = {S}_{1} \times [ {E}_{SAP} - ( k * a ) ] \times [ {E}_{SAP} < ( k * a ) ] + [ {E}_{SAP} + {I}_{2} ]$$

As a measure of the robustness of the model, Gaussian noise (mean: 0, sd: 0.02) was added to the energy values for both GIII and AMP, and the two-phase regression analysis was run again in the same way. This was repeated 5,000 times, collecting fit parameters (slope of the first segment, intercept of the second segment, and breakpoint value).

### Verification of Data Comparison

Proof of concept for area-modulated stimuli was provided in a study in which four regions of the visual field were tested. In a perimetry test, one needs to be able to measure sensitivity/threshold at multiple locations to obtain an overview of patterns of sight loss across the visual field, but also to maintain good diagnostic performance. There is no specific number of locations that should be tested, and the number of test locations varies from test to test. Here, we provide proof-of-concept that a) area-modulated stimuli can still distinguish between glaucomatous and healthy visual fields when the test is expanded to test many more visual field locations, and b) the disease signal is still greater for area-modulated stimuli than for conventional luminescent-modulated or contrast-modulated stimuli when many more locations are tested.

In this study, energy thresholds were measured for four different stimulus types on five separate occasions, over five visits, within 11 weeks.

It is generally assumed in the literature that this timescale is too short for disease progression to be measurable, therefore the five measures for each stimuli over this period could be considered as repeat measurements of the same sensitivity/threshold level. In this regard, we emphasise the findings from two stimulus forms: 1) the area-modulated stimulus (“A-stimulus”) and 2) the conventional Goldmann III stimulus (“GIII-stimulus”) and the data analysis from the fifth visit, to minimise any possible learning effect.

### Apparatus

Experiments were programmed in MATLAB (version 2014b; The MathWorks, Inc., Natick, MA) with the CRS toolbox (v.1.27 Cambridge Research Systems, Rochester, UK) and performed on a γ-corrected 25″ OLED monitor (SONY PVM-A250 Trimaster El, resolution: 1929 1080 pixels, frame rate: 60 Hz, refresh rate: 120 Hz). The luminance of the background on which stimuli were displayed was 10 cd/m2. A cross was present in the centre of the screen throughout the experiments, as a fixation point.

### Stimuli

Stimuli were of constant contrast and duration (200 ms), varying only in area with participant responses. GIII-stimuli was of constant area (0.43 deg diameter) and duration (200 ms), varying only in contrast with participant responses. At each visit, energy thresholds were measured at each test location with each stimulus form. Within a single test, the order of stimulus presentations to each location was interleaved. Eighteen test locations were chosen, including those reported as being an optimised subset to enable good diagnostic performance with conventional perimetry with Goldmann III stimuli.

### Procedure

Participants were asked to keep their eye steady and watch the fixation point at all times throughout the test as stimuli were presented to their peripheral vision. A 1 up/1 down adaptive staircase procedure was used in the determination of threshold at each test location and for each stimulus form. Stimulus energy was increased by 0.5 log units until the first reversal, 0.25 log units until the second reversal, 0.1 log units until the third reversal, and 0.05 log units until the fourth reversal. Threshold was taken as the mean of energy values at the third and fourth reversal. Participants were asked to press a response pad when they detected a stimulus presentation anywhere in their peripheral vision. Catch trials, in the form of blank (false positive) and highly suprathreshold (false negative) presentations were included as tests of reliability. The order of tests (A-stimulus OR GIII-stimulus) was randomised at each visit and between participants. One measurement of threshold for each stimulus form was performed at each visit, following initial practice sessions to minimise any potential fatigue effect.

### Participants

### Glaucoma

Fifteen patients with glaucoma (median [IQR] age: 69.5 [67.5, 72.1] years) and five age-similar healthy controls (median [IQR] age: 71.9 [67.6, 72.5] years) were recruited and tested. All glaucoma patients had a repeatable visual field defect (measured with a Humphrey Field Analyzer [HFA II, Carl Zeiss Meditec, Dublin, CA], SITA-Standard 24-2 program) ranging from near normal (‘borderline’; Glaucoma Hemifield Test) to ‘advanced’ (Hodapp-Parish-Anderson grading scale). Median [IQR] ‘mean deviation’ (MD) was −4.88 [−6.78, −2.62] dB. The visual field was normal (‘within normal limits’; Glaucoma Hemifield Test) on the same test. Median [IQR] MD for controls was +0.46 [−1.26, +0.92] dB. One eye of each participant was tested, and the same eye was tested for each visit and with each stimulus form.

Participants had no ocular or systemic disease or disorder and/or medication known to affect visual performance (except for glaucoma in the glaucoma patient group). Apart from one patient who had undergone a trabeculectomy procedure in the test eye seven years prior to the study (typically assumed to be stable after this timescale), no participant had undergone any ocular surgery, with the exception of uncomplicated cataract surgery.

Intraocular pressure (IOP) was <21 mmHg at each visit in all participants. None of the controls had any first-degree relatives with glaucoma nor a history of elevated IOP. Best-corrected visual acuity was ≥6/9 in the test eye and no significant media or corneal opacity (≤NO3, NC3, C3, and/or P3, LOCS III.23). A full refraction was conducted prior to experimental tests; refractive error for all participants was between +6.00DS and −6.50DS and astigmatism was <3.50DC. If a participant had a history of cataract surgery and their pre-surgical refractive error was known to be higher than these limits, they were excluded from participation in the study. Full aperture trial lenses were used throughout experiments (to correct refractive error for a viewing distance of 30 cm), mounted in a half-eye trial frame. The non-test eye was occluded with an eye patch.

### Data Analysis

Data for the fifth visit (to minimise any learning effect) were averaged separately across glaucoma patients and across healthy controls for each test location, giving a mean threshold value for each location in the glaucoma and control groups.

Disease signal was calculated for a given location by subtracting the mean threshold for healthy controls for the mean threshold from glaucoma patients. This analysis was performed separately for thresholds measured with the A-stimulus and for those measured with the GIII-stimulus.

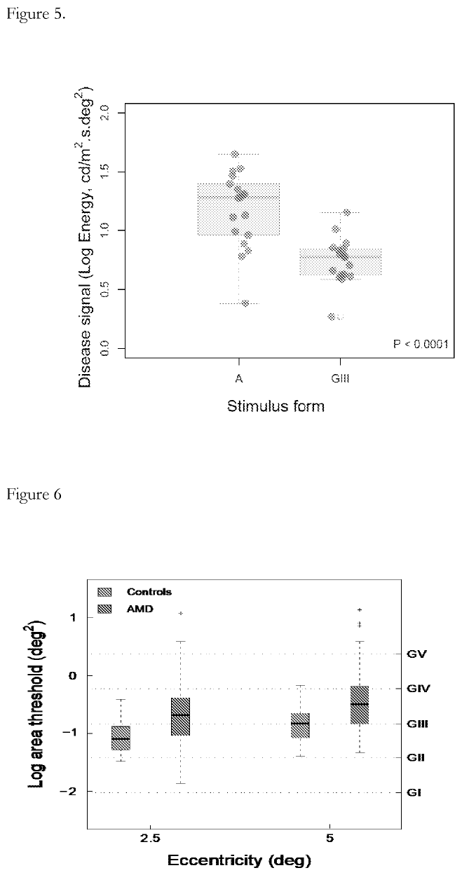

Disease signal (difference in threshold between glaucoma patient and healthy control cohorts) was greater than 0 for all locations for both the A-stimulus and the GIII-stimulus. Disease signal was greater for the A-stimulus than for the GIII-stimulus, and this was statistically significant (t=4.88, df=27.69; P<0.0001). These data are represented in FIG. 5. An ad-hoc analysis of the 1st, 2nd, 3rd, and 4th visit data revealed the same result; disease signal for all locations >0; all P<0.001).

The data show that area-modulated stimuli can still distinguish between glaucomatous and healthy visual fields when the test is expanded to many more visual field locations. They also demonstrate that disease signal is still significantly greater for area-modulated stimuli than for conventional contrast-modulated stimuli when many more locations are tested.

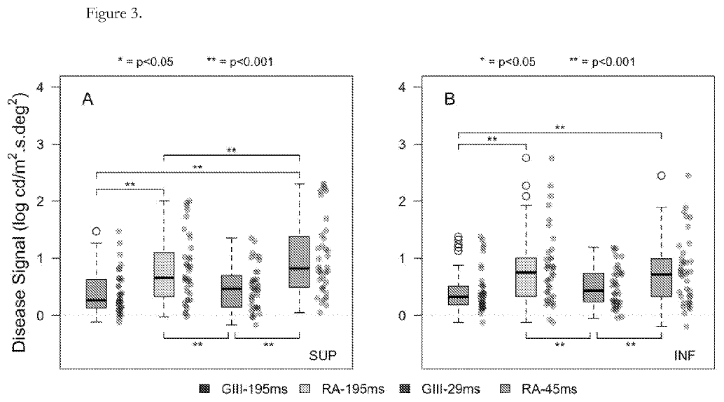

From FIG. 3 it can be seen that the disease signal is greater when a luminance-modulating stimulus has an area that is smaller than Ricco's area and duration that is shorter than the critical duration.

### Participants

### AMD

Twenty participants with dry AMD (mean age, 74.6 years) and 20 healthy controls (mean age, 67.8 years) were recruited and performed a range of custom gaze-contingent microperimetry tests. An area-modulation test with a stimulus of fixed luminance was used to generate localised estimates of Ricco's area (RA) at 2.5° and 5° eccentricity along the 0°, 90°, 180°, and 270° meridians. Contrast thresholds were measured at the same test locations for stimuli of six different durations (1-15 frames, 3.7-190.4 ms) with a Goldmann III stimulus (GIII, 0.43° diameter) and fixed-area stimuli equal in area to the measured RA estimates. Iterative two-phase regression analysis was used to estimate the upper limit (critical duration) of complete temporal (using GIII stimulus) and spatiotemporal summation (using the RA stimulus).

### Results

### Spatial Summation

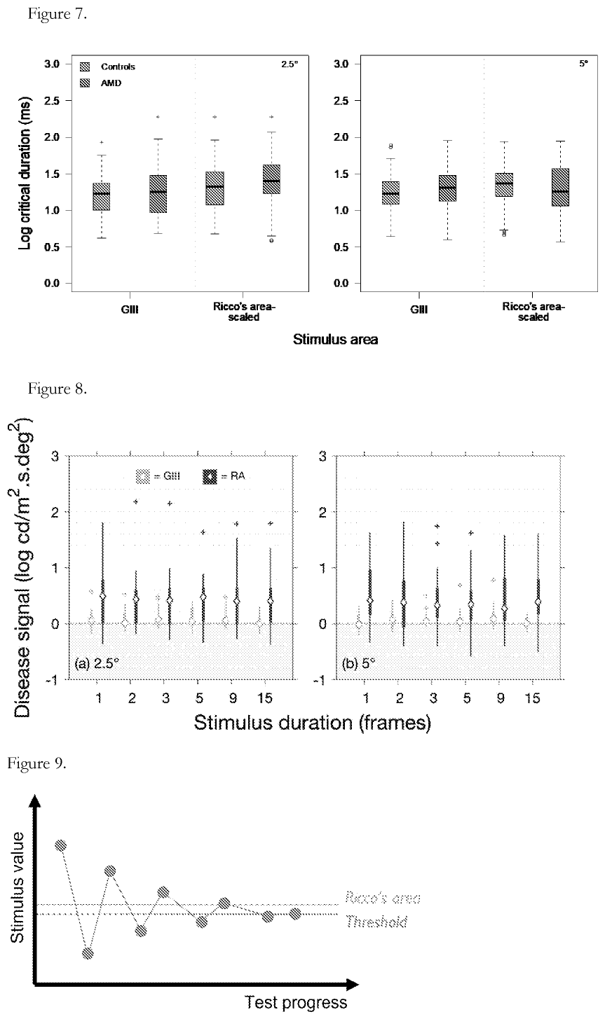

Estimates of Ricco's Area (RA) were significantly larger in AMD participants, compared to healthy controls at all test locations (all P<0.05, Wilcoxon rank sum test, FIG. 7). In agreement with previous studies, statistically significant enlargements in RA estimates were observed with increasing visual field eccentricity (2.5° vs) 5° for both control and AMD participant groups (control: P<0.001, AMD: P=0.03, Wilcoxon rank sum test).

### Temporal Summation

While critical duration values were longer in AMD participants compared to healthy controls with the GIII stimulus at 2.5° (AMD: 17.9 ms; IQR, 9.3-30.4, Controls: 17.0 ms; IQR, 10.1-23.5) and 5° (AMD: 20.6 ms; IQR, 13.2-30.4, Controls: 17.0 ms; IQR, 12.1-25.0), these differences were not statistically significant (all P>0.05, Wilcoxon rank sum test). Similarly, the critical duration was longer in AMD participants at 2.5° with stimuli scaled to localised spatial summation characteristics (AMD: 25.3 ms; IQR, 17.0-42.0, Controls: 21.1 ms; IQR, 11.8-33.9), but this also failed to reach statistical significance (P=0.33). Conversely, a non-significant (P=0.36) shortening of the critical duration was observed at 5° when examined using RA-scaled stimuli (AMD: 18.2 ms; IQR, 11.2-38.2, Controls: 23.4 ms; IQR, 15.6-32.7). Boxplots reporting critical duration values at 2.5° and 5° tested under both stimulus conditions for healthy control and AMD participants are shown in FIG. 7. Disease signal (log cd/m2·s·deg2) was higher with RA-sized stimuli compared to the GIII stimulus for all stimulus durations at 2.5° and 5° visual field eccentricity (P<0.001, FIG. 9). This preliminary AMD data implies critical duration values are likely to be longer in AMD patients compared to healthy controls.

In summary, spatial summation is altered in early-intermediate dry-AMD but there was no statistically significant change in temporal summation in AMD compared to controls were found.

The sensitivity of perimetric strategies to detect and monitor functional changes in AMD may be markedly improved if stimuli capable of probing alterations in spatial summation are used. Our test strategy (demonstrated in this study) used a stimulus of constant luminance (equal to the luminance contrast threshold expected for a stimulus equal or near in area to Ricco's area) and duration (□1200 ms), that varied in area in line with participant responses.

### Testing of the Invention Protocol

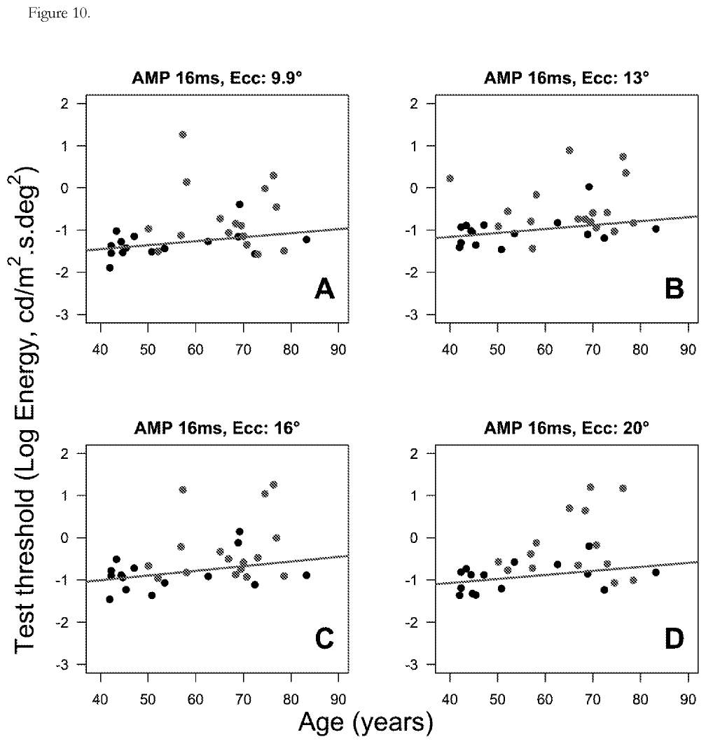

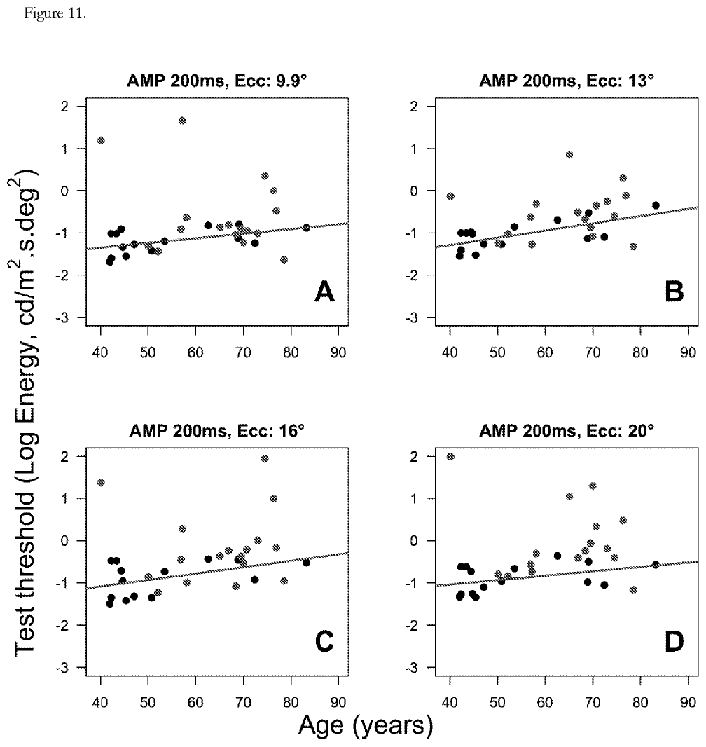

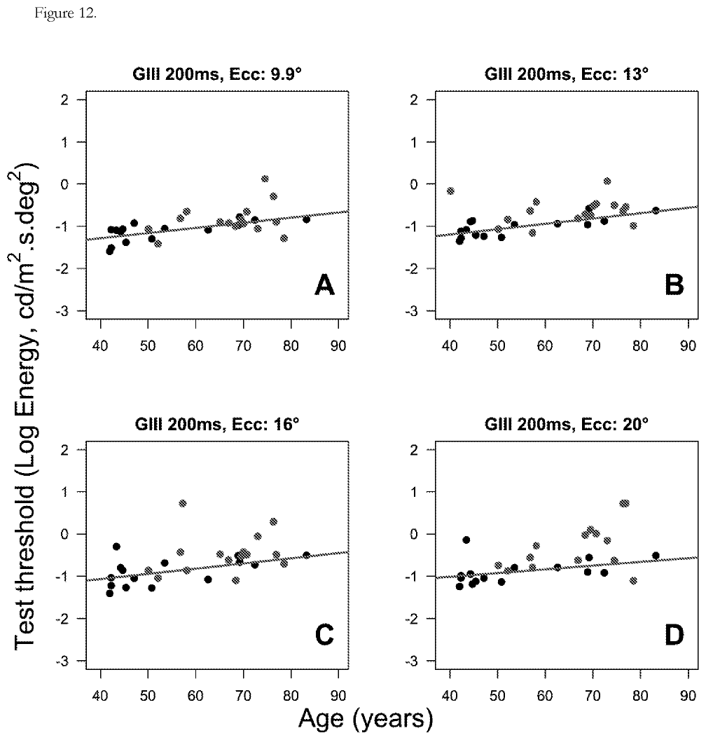

Using the method of the invention, 17 patients were tested to determine if the method described herein outperformed conventional testing using Goldmann Methodology. Graphs A-D in FIGS. 10-13 show threshold (limit of vision) for healthy controls (black/dark dots) and glaucoma patients (blue/light dots). A regression line was plotted through the control data only and describes how the visual limits change as a function of age (x axis). The way the y-axis works is that higher numbers=worse vision. So, glaucoma patients' data should be higher than what would be predicted for someone healthy of their age, i.e., the further the blue/light dots are away from the black line (in a vertical direction) the worse their visual field sensitivity. In FIGS. 10-13, 9.9° refers to a location that is 9.9 degrees of visual angle away from central fixation, 13° refers to a location that is 13 degrees of visual angle away from central fixation, 16° refers to a location that is 16 degrees of visual angle away from central fixation, and 20° refers to a location that is 20 degrees of visual angle away from fixation. Data obtained using the method of the invention is shown in FIGS. 10 and 11 where, FIG. 10 shows data for a single stimulus type: fixed luminance, variable area, fixed duration at 16 ms and within the critical duration (we call this AMP 16 ms—area-modulated stimulus @ 16 msec); and FIG. 11 shows data also for a single stimulus type: fixed luminance, variable area, fixed duration near 200 ms and outside the critical duration (we call this AMP 200—area-modulated stimulus @ 200 msec);

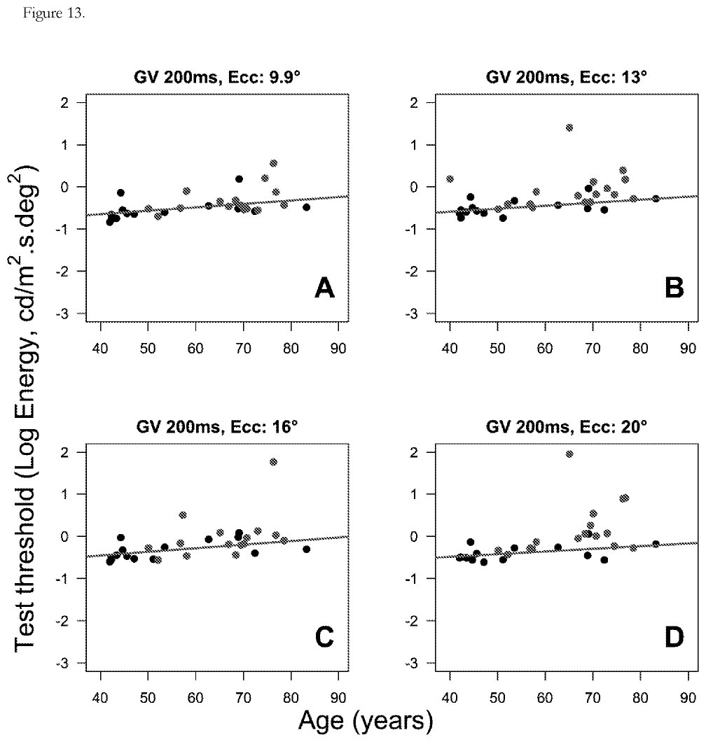

The data obtained in FIGS. 12 and 13 were obtained using conventional Goldmann testing techniques. FIG. 12 shows data for a single stimulus type: fixed area (Goldmann III or “GIII”), variable luminance, fixed duration near 200 ms and outside the critical duration (this is the current clinical reference standard); and FIG. 13 shows data for a single stimulus type: fixed area (Goldmann V or “GV”), variable luminance, fixed duration near 200 ms and outside the critical duration.

The data clearly shows the testing undertaken for FIGS. 10 and 11 is far more discriminatory in that it scales the patients and so identifies those whose vision is declining. In contrast the tests used to obtain the data in FIGS. 12 and 13 is far less discriminatory in that all the patients produce test results that hover around the regression line and so appear normal.

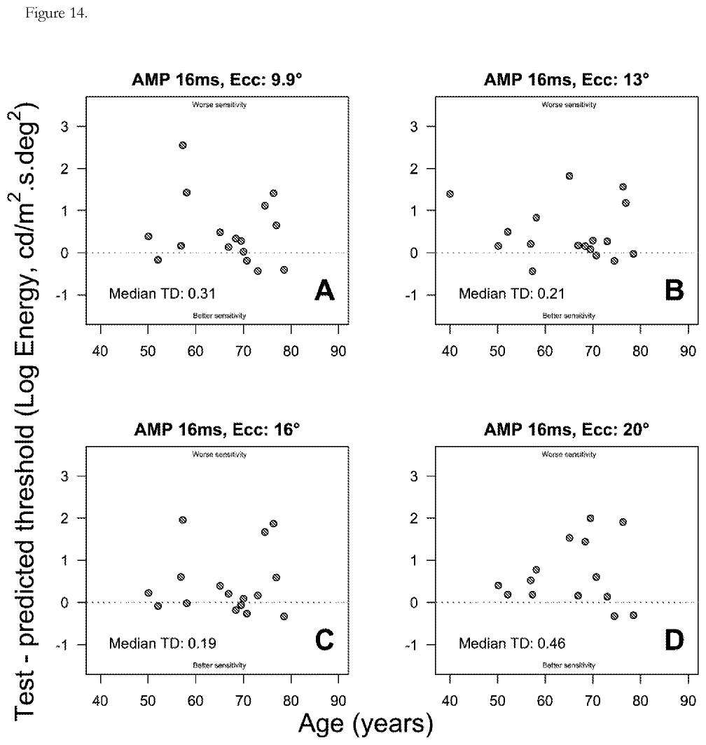

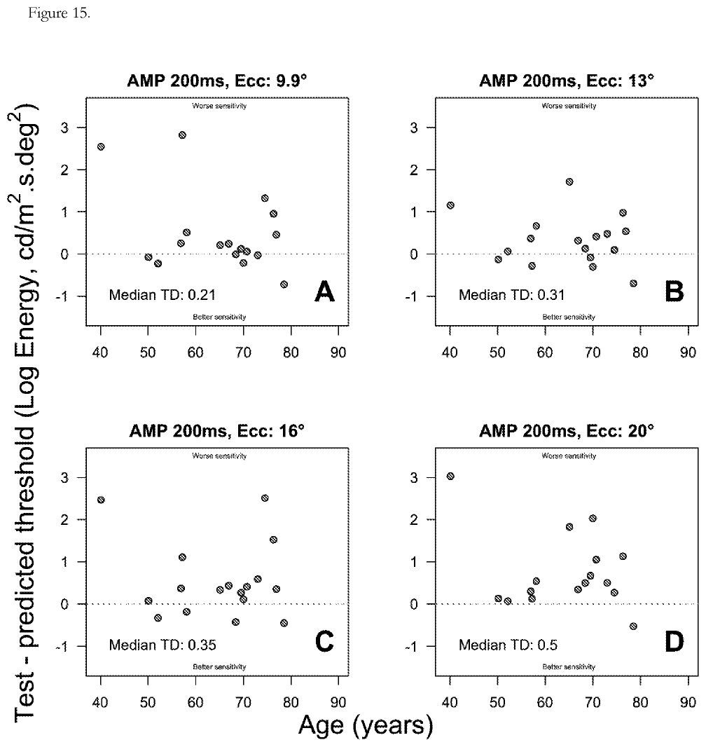

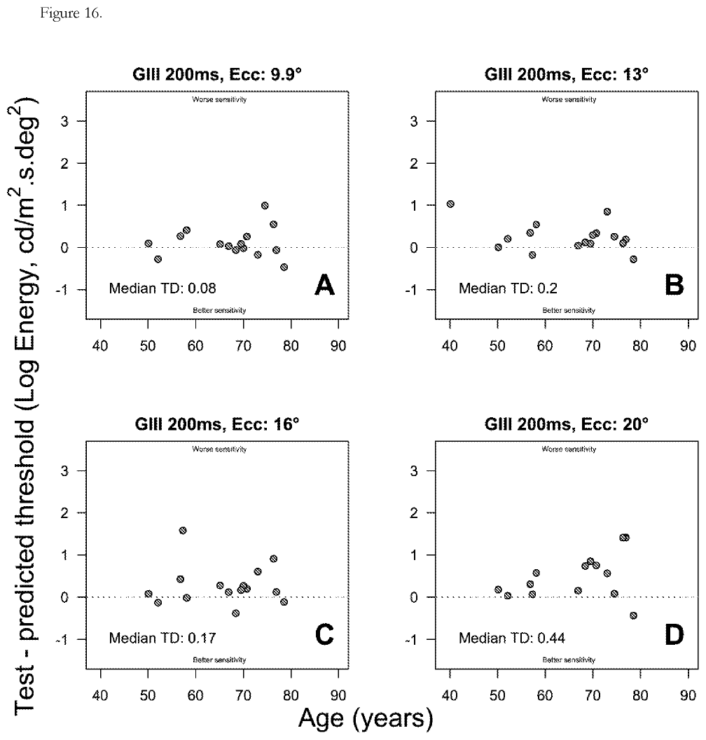

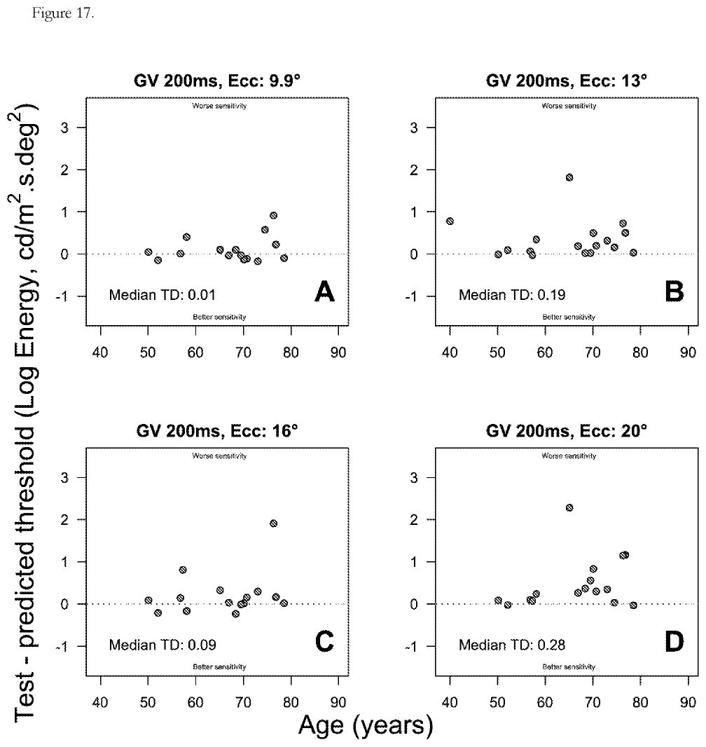

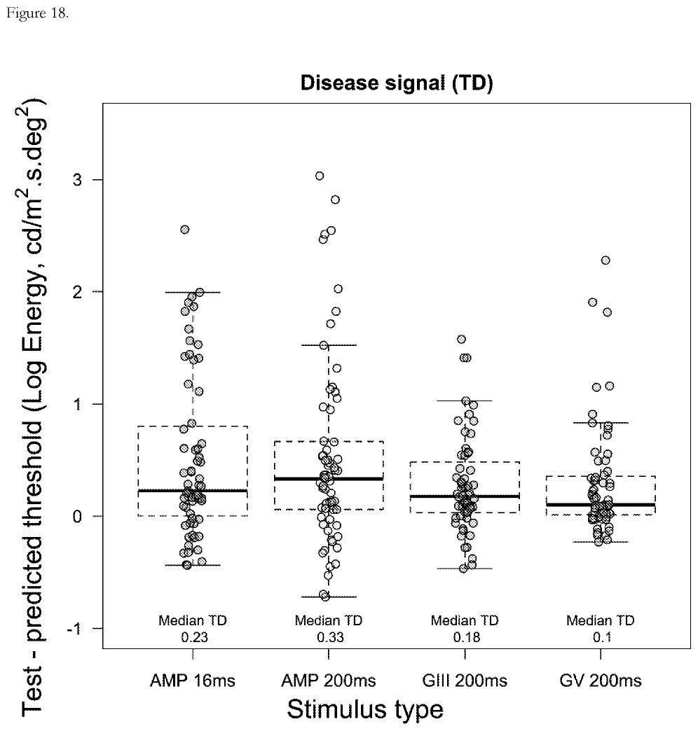

FIGS. 14-17 shows how much each glaucoma patient's threshold differs from the predicted value from their age (i.e., “difference from an age-matched control” or “disease signal”). In these figures, if data are clumped around the horizontal line, threshold is much the same as that of an age-matched control; if they are above the line, the threshold is higher (i.e., vision is worse) than an age-matched control; if they are below the line, the threshold is lower (i.e., vision is better) than an age-matched control. FIGS. 14-17 show this analysis for each of the test locations and for each of the 4 stimulus types as described above in FIGS. 10-13. It can be seen that data obtained using the conventional Goldmann methods of testing approximates more closely to the control age-matched horizontal line thus being less able to distinguish early stages of disease. Whereas, in contrast, data obtained using the methods of the invention, AMP perimetry, are more scattered about the control age-matched horizontal line, thus being able to distinguish early stages of disease.

FIG. 18 shows the pooled data of FIGS. 10-13 presented as boxplots. These boxplots show disease signal for each of the stimulus types and for each visual field location. Disease signal is the distance between the points and the horizontal lines in the plots, in particular the thick horizontal line representing the median signal. In other words, it is how much each individual location/patient differs from what would be predicted (the median) for an age-similar normal test. This is a standard method of measuring the magnitude of disease with a particular stimulus in the clinical setting.

In FIG. 18, it can be seen that the median line is higher for the two area-modulated stimuli (AMP) than for the two luminance-modulated stimuli (GIII or GV), indicating AMP will be more sensitive/discriminatory for determining a disease signal. Further, FIG. 18 also shows that AMP16 and AMP200 (i.e., area modulated stimuli) show a greater disease signal (i.e., greater vertical distance from the median disease signal, indicating with greater reliability the possibility of disease) than the standard GIII & GV at all test locations.

Notably, a test that discriminates glaucoma from normal, requires a positive disease signal (i.e., median above 0). If a test were unable to discriminate between glaucoma and normal, it would have a median disease signal at 0, with perhaps some variance in the data around that point. Here, in FIG. 18, we show two things: 1) the median is greater than 0, indicating that the test can discriminate between glaucoma and normal, and 2) the median for area-modulated stimuli is greater than that of the luminance-modulated stimuli, demonstrating the test can not only discriminate between glaucoma and normal but it can pick up disease signal at an earlier stage in disease progression, thus help to safeguard against future sight loss.

1. A method for measuring visual field sensitivity comprising:
i) presenting to a subject a fixed-luminance stimulus of a first size at one or more location(s) in the subject's visual field and recording the response of the subject to the said stimulus at each one or more locations in the visual field;
ii) presenting to the same subject a fixed-luminance stimulus of a second, different size at the one or more location(s) in the subject's visual field and recording the response of the subject to the said stimulus at each one or more location(s) in the visual field;
iii) using the subject's responses in parts i)-ii) to determine a threshold for the detection of said fixed-luminance area-modulated stimuli at said one or more locations;
iv) comparing the determined threshold of part iii) for said subject with either, or both, of a threshold for the detection of said fixed-luminance area-modulated stimuli in an age-matched normal group or of a threshold(s) for the detection of said fixed-luminance area-modulated stimuli in at least one prior determined baseline to identify any change in a visual field sensitivity; and
v) where a change in visual field sensitivity is found, using it as an indicator of a change in visual pathway function or disease.

2. The method according to claim 1 wherein said luminance of said fixed-luminance stimulus is equal to or greater than the luminance of a stimulus equivalent in size to Ricco's area at threshold for age-similar healthy individuals at an equivalent visual field location.

3. The method according to claim 1 wherein said fixed-luminance stimulus at each location is presented for a fixed duration.

4. The method according to claim 1 wherein said fixed-luminance stimulus at each location is equal to or shorter than a critical duration or shorter than and scaled to the critical duration for age-similar healthy individuals at an equivalent visual field location.

5. The method according to claim 4 wherein the duration of the fixed-luminance stimuli is shorter than the critical duration.

6. The method according to claim 3 wherein said fixed-luminance stimulus duration is modulated so that the stimulus of part i) of a first fixed size is presented for a fixed first duration but the stimulus of part ii) of a second, different fixed size is presented for said fixed first duration or a fixed second different duration.

7. (canceled)

8. The method according to claim 6 where, when stimuli of different durations are used, temporal summation and/or critical duration is tested.

9. The method according to claim 1 wherein the size of the area stimuli is equal to or smaller than Ricco's area.

10. The method according to claim 1 wherein the said one or more locations are in close proximity, whereby spatial summation is tested.

11. The method of claim 10 whereby Ricco's area is tested.

12. The method according to claim 1 wherein said fixed-luminance stimulus is a spot or circular shaped stimulus.

13. The method according to claim 1 wherein the fixed-luminance stimulus is converted into an energy value using the following formula:
$$\begin{matrix}E = A \times D \times L or \\ E = A \times D \times ML \times {10}^{( \frac{- dB}{10} )}\end{matrix}$$
Where A=stimulus area, D=presentation duration, and L=a stimulus luminance calculated from the dB value associated with the HFA using the following equation (eq 3):
$$L = ML \times {10}^{( \frac{- dB}{10} )}$$
Where ML=maximum luminance, thus combining the afore two equations to convert from dB to E (eq 4):
$$E = A \times D \times ML \times {10}^{( \frac{- dB}{10} )}$$

14. The method according to claim 1 wherein the at least one prior determined baseline data is converted into a stimulus energy value using the following formula, before step v) is performed,
$${E}_{AMP} = {S}_{1} \times [ {E}_{SAP} - k ] \times [ {E}_{SAP} < k ) ] + [ {E}_{SAP} + {I}_{2} ]$$
where E_{SAP} is SAP sensitivity converted to energy using Equations 3 and 4 of claim 13, E_{AMP} is the equivalent energy threshold for AMP, S₁ is the slope of the first segment in the two-phase model, I₂ is the intercept of the second segment in the two-phase model, and k is the energy value for a stimulus of equivalent area to Ricco's area and the luminance level of this area at threshold.

15. The method according to claim 14 wherein the at least one prior determined baseline data is converted into a stimulus energy value using the following formula, before step v) is performed,
$${E}_{AMP} = {S}_{1} \times [ {E}_{SAP} - ( k * a ) ] \times [ {E}_{SAP} < ( k * a ) ] + [ {E}_{SAP} + {I}_{2} ]$$
where E_{SAP} is SAP sensitivity converted to energy using Equations 3 and 4 of claim 13, E_{AMP} is the equivalent energy threshold for AMP, S₁ is the slope of the first segment in the two-phase model, I₂ is the intercept of the second segment in the two-phase model, and k is the energy value for a stimulus of equivalent area to Ricco's area and the luminance level of this area at threshold, and we introduce a parameter a, to adjust the contrast level for the stimulus equivalent in area to Ricco's area.

16. The method according to claim 1 wherein step v) includes statistical analysis to determine the probability that a deviation in said threshold at a given location, or a number of locations, is representative of said change.

17. The method according to claim 1 wherein the fixed-luminance stimuli are presented at different locations, in close proximity, the fixed-luminance stimuli are presented for different durations, or both, whereby detected abnormalities in spatial and/or temporal summation, respectively, are indicators of a disease.

18. The method according to claim 17 wherein said disease is glaucoma or age-related macular degeneration (AMD).

19. An instrument for measuring visual field sensitivity comprising:
i) at least one light stimulus emitter adapted to emit fixed-luminance stimuli of variable sizes for stimulating, at least, a first location in a subject's visual field, wherein said light stimulus emitter is moveable relative to the subject's visual field, whereby a further fixed-luminance light stimulus of a first or different size can be emitted to stimulate said first or a different location in the same subject's visual field;
ii) a recording device for recording the subject's response to said fixed-luminance light stimulus at said first or different location in the subject's visual field;
iii) a processor adapted for capturing the subject's response to said light stimuli at each, or a selected one or more, location(s) in said subject's visual field and adjusting said light stimulus emitter to emit a further fixed-luminance stimulus of a different size or targeted to a different location and then using the subject's responses to determine a threshold for the detection of said fixed-luminance area-modulated stimuli at said one or more locations;
iv) the processor of iii) or a further processor adapted for comparing the threshold of iii) with either, or both, the threshold in an age-matched normal group or the threshold in a prior determined baseline to identify a change in visual field sensitivity and where a change in visual field sensitivity is found, using it as an indicator of a change in visual pathway function or disease; and
v) a display device for displaying the results of step iii) and/or iv) to a user.

20. The instrument according to claim 19 wherein said light stimulus emitter comprises i) at least one light stimulus emitter that moves relative to the subject's visual field or ii) said light stimulus emitter comprises multiple light stimulus emitters each emitting fixed-luminance stimuli, sequentially or concurrently, and arranged to target different locations in a subject's visual field.

21. The instrument according to claim 19 wherein said emitter is adapted to emit a fixed-luminance stimulus equal to or greater than the luminance of a stimulus equivalent in size to Ricco's area at threshold for age-similar healthy individuals at an equivalent visual field location.

22. The instrument according to claim 19 wherein said emitter is adapted to emit a fixed-luminance stimulus for a fixed duration.

23. The instrument according to claim 22 wherein said emitter is adapted to emit a fixed-luminance stimuli for a fixed duration equal to or shorter than a critical duration or shorter than and scaled to the critical duration for age-similar healthy individuals at an equivalent visual field location.

24. The instrument according to claim 23 wherein the duration of the stimuli is shorter than the critical duration.

25. The instrument according to claim 19 wherein said emitter is adapted to emit a fixed-luminance stimulus of any selected duration.

26. The instrument according to claim 19 wherein the size of the area stimulus is equal to or smaller than Ricco's area.

27. The instrument according to claim 19 wherein said emitter is adapted to emit fixed-luminance stimuli of different sizes and different durations.

28. The instrument according to claim 19 wherein the instrument includes a processor for converting the stimulus into an energy value using the following formula:
$$\begin{matrix}E = A \times D \times L or \\ E = A \times D \times ML \times {10}^{( \frac{- dB}{10} )}\end{matrix}$$
Where A=stimulus area, D=presentation duration, and L=a stimulus luminance calculated from the dB value associated with the HFA using the following equation (eq 3):
$$L = ML \times {10}^{( \frac{- dB}{10} )}$$
Where ML=maximum luminance, thus combining the afore two equations to convert from dB to E (eq 4):
$$E = A \times D \times ML \times {10}^{( \frac{- dB}{10} )}$$

29. The instrument according to claim 28 wherein the instrument includes a processor for converting the prior determined baseline data into a stimulus energy value using the following formula, before step iv) is performed
$${E}_{AMP} = {S}_{1} \times [ {E}_{SAP} - k ] \times [ {E}_{SAP} < k ) ] + [ {E}_{SAP} + {I}_{2} ]$$
where E_{SAP} is SAP sensitivity converted to energy using Equations 3 and 4, E_{AMP} is the equivalent energy threshold for AMP, S₁ is the slope of the first segment in the two-phase model, I₂ is the intercept of the second segment in the two-phase model, and k is the energy value for a stimulus of equivalent area to Ricco's area and the luminance level of this area at threshold.

30. The instrument according to claim 28 wherein the instrument includes a processor for converting the prior determined baseline data into a stimulus energy value using the following formula, before step iv) is performed
$${E}_{AMP} = {S}_{1} \times [ {E}_{SAP} - ( k * a ) ] \times [ {E}_{SAP} < ( k * a ) ] + [ {E}_{SAP} + {I}_{2} ]$$
where E_{SAP} is SAP sensitivity converted to energy using Equations 3 and 4 in claim 28, E_{AMP} is the equivalent energy threshold for AMP, S₁ is the slope of the first segment in the two-phase model, I₂ is the intercept of the second segment in the two-phase model, and k is the energy value for a stimulus of equivalent area to Ricco's area and the luminance level of this area at threshold and we introduce a parameter a, to adjust the contrast level for the stimulus equivalent in area to Ricco's area.

31. The instrument according to claim 19 wherein step iv) includes statistical analysis to determine the probability that a deviation at a given location, or a number of locations, is representative of said change in visual pathway function or disease.

32. The method according to claim 1, further comprising:
iii) repeating step ii) for one or more iterations using one or more of the further fixed-luminance stimuli but of one or more further sizes;
iv) using the subject's responses in parts i)-iii) to determine a threshold for the detection of said fixed-luminance area-modulated stimuli at said one or more locations;
v) comparing the determined threshold of part iv) for said subject with either, or both, the threshold in an age-matched normal group or the threshold(s) in at least one prior determined baseline to identify any change in visual field sensitivity; and
vi) where a change in visual field sensitivity is found, using it as an indicator of a change in visual pathway function or disease.

33. The method according to claim 32, further wherein the said stimulus duration is the same as the duration of part i) or part ii) or a further different fixed duration.

34. The method according to claim 32, wherein when stimuli of different durations are used, temporal summation and/or critical duration is tested.
